(19) United States
(12) Patent Application Publication
(10) Pub. No.: US 2006/0166288 A1
(43) Pub. Date: Jul. 27, 2006

(54) Assay for the screening of compounds acting through erbb-2

(75) Inventor: Daniel Mark Hickinson, Macclesfield (BE)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/532,152

(22) PCT Filed: Oct. 28, 2003
(86) PCT No.: PCT/GB03/04655

§ 371 Date: Apr. 20, 2005

(30) Foreign Application Priority Data
Oct. 31, 2002 (GB) 0225282.3

(51) Int. Cl. G01N 33/574

(52) U.S. Cl. 435/7.23

(57) ABSTRACT

The invention provides a cellular proliferation assay for a compound acting through erbB-2 which comprises: i) a cell comprising erbB-2 and erbB-3 and said cell is responsive to ligand stimulated cell proliferation under conditions suitable for cell proliferation; ii) a first ligand which is a ligand for erbB-3 capable of inducing proliferation by the cell; iii) mixing i) and ii) in the presence and absence of compound; iv) measuring whether the compound has any effect on reducing cell proliferation.

This invention relates to a cellular proliferation assay for a compound acting through erbB-2 wherein the cell is responsive to ligand stimulated cell proliferation.

New therapeutic approaches to cancer are needed. The receptor tyrosine kinases are of particular importance in the transmission of mitogenic signals that initiate cellular replication. These large glycoproteins, which span the plasma membrane of the cell possess an extracellular binding domain for their specific ligands (such as Epidermal Growth Factor (EGF) for the EGF Receptor). Binding of ligand results in the activation of the receptor's kinase enzymatic activity that is encoded by the intracellular portion of the receptor. This activity phosphorylates key tyrosine amino acids in target proteins, resulting in the transduction of proliferative signals across the plasma membrane of the cell.

It is known that the erbB family of receptor tyrosine kinases, which include EGFR, erbB-2, erbB-3 and erbB-4, are frequently involved in driving the proliferation and survival of tumour cells (reviewed in Olayioye et al EMBO J., 2000, 19, 3159). One mechanism in which this can be accomplished is by overexpression of the receptor at the protein level, generally as a result of gene amplification. This has been observed in many common human cancers (reviewed in Klapper et al., Adv. Cancer Res., 2000, 77, 25).

As a consequence of the mis-regulation of one or more of these receptors (in particular erbB-2), it is widely believed that many tumours become clinically more aggressive and so correlate with a poorer prognosis for the patient (Brabender et al, Clin. Cancer Res., 2001, 7, 1850; Ross et al, Cancer Investigation, 2001, 19, 554, Yu et al., Bioessays, 2000, 22.7, 673). In addition to these clinical findings, a wealth of pre-clinical information suggests that the erbB family of receptor tyrosine kinases are involved in cellular transformation. This includes the observations that many tumour cell lines overexpress one or more of the erbB receptors and that EGFR or erbB-2 when transfected into non-tumour cells have the ability to transform these cells. This tumourigenic potential has been further verified as transgenic mice that overexpress erbB-2 spontaneously develop tumours in the mammary gland. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 12, 6550). Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565). In addition to this pre-clinical data, findings using inhibitory antibodies against EGFR and erbB-2 (c-225 and trastuzumab respectively) have proven to be beneficial in the clinic for the treatment of selected solid tumours (reviewed in Mendelsohn et al, 2000, Oncogene, 19, 6550-6565).

The search for new drugs frequently involves a “screening cascade”. This cascade often starts with an in vitro assay and ultimately extends into in vivo assays. More meaningful data is often obtained from in vivo assays, but at the sacrifice of throughput such that it is not usually practicable to put a large number of compounds through an in vivo assay. It is usual to screen compounds in higher throughput assays and take forward only the more promising leads to the next assay in the screening cascade for optimal efficiency. A highly desirable assay which fits between in vitro and in vivo assays is a cell proliferation assay.

It is known that non-neoplastic epithelial cells like H16N-2 can respond in a proliferative manner to stimulation with either EGF or heregulin (Ram, G. R. and Ethier, S. P. (1996) Cell Growth and Differentiation, 7, 551-561). However there is presently no known cell based assay for compounds acting through erbB-2 that uses a ligand stimulated proliferation assay to measure cytostatic and cytotoxic potency. There is a need for such an assay because of the desire to distinguish between a compound that specifically inhibits cell proliferation through through erbB-2 (real hits) and those compounds inhibiting cell growth through non-specific toxicity caused by the compound (false hits).

One aspect of the invention provides a cellular proliferation assay for a compound acting through erbB-2 which comprises:

i) a cell comprising erbB-2 and erbB-3 and said cell is responsive to ligand stimulated cell proliferation under conditions suitable for cell proliferation;

ii) a first ligand which is a ligand for erbB-3 capable of inducing proliferation by the cell;

iii) mixing i) and ii) in the presence and absence of compound;

iv) measuring whether the compound has any effect on reducing cell proliferation

The cell may be recombinant or non-recombinant but if recombinant they should be engineered lines to express the receptors at low/normal levels to gain ligand responsiveness. “Mixing” simply means that the components are brought together into a homogeneous mixture, continuous mixing is not required. Measurement of cell proliferation may be by any suitable means; Example 1 below exemplifies one suitable method.

Preferably the cellular proliferation assay comprises the addition of a control wherein the compound is tested in parallel in the absence of ligand to detect non-specific cell toxicity. This has the advantage of being able to distinguish between a compound that specifically inhibits cell proliferation through through erbB-2 (real hits) and those compounds inhibiting cell growth through non-specific toxicity caused by the compound (false hits).

Preferably the cellular proliferation assay comprises the addition of a second assay which is an assay for measuring whether the compound has activity through EGFR which comprises:

i) a cell comprising erbB-2, erbB-3 and EGFR under conditions suitable for cell proliferation;

ii) a second ligand which is a ligand for EGFR capable of inducing proliferation by the cell;

iii) mixing i) and ii) in the presence and absence of compound;

iv) measuring whether the compound has any effect on reducing cell proliferation.

This is advantageous because it enables the ability to profile the activity of compounds potentially acting through erbB-2 and EGFR. Note that for some pharmaceutical purposes it is desirable to have compounds that act through both targets (dual specificity). It is also contemplated that combinations of individual compounds may also be tested.

Preferably the cellular proliferation assay provided herein endogenously expresses EGFR and/or erbB-2 and/or erbB-3, more preferably EGFR, erbB-2 and erbB-3. “Endogenously” means that the cell has not been recombinantly engineered to express a particular receptor.

Preferably the first ligand is ‘a member of the neuregulin family of ligands, preferably heregulin Most preferably the first ligand is heregulin β1.

Preferably the second ligand is EGF.

In one embodiment a preferred cell is an immortalised normal epithelial H16N-2 cell.

In another embodiment a preferred cell is a MCF-7 cell MCF-7 cells are available from the ATCC. MCF-7 cells are neoplastic however they have a ‘normal’ erbB receptor expression profile.

“Cell proliferation” means increase in cell number through a process of cell division. Suitable conditions for cell proliferation are known in the art through provision of appropriate media, incubation conditions etc. Example 1 below provides one set of conditions for a particular cell line.

“Ligand stimulated cell proliferation” means that cell proliferation is driven through the activation of endogenous receptor by the addition of an exogenous ligand.

“Reduces cell proliferation” means that there is a statistically significant (95% confidence, preferably 99% confidence) reduction in cell number compared with control

“Control” means a reference measurement against which a test result is compared to reduce experimental errors.

“A compound acting through erbB-2” means that proliferation is reduced as a consequence of the inhibition of an activity associated with erbB-2. The invention is particularly useful for detecting compounds which are kinase inhibitors, for example ATP mimetics. Note that the kinase domain of erbB-3 bears little homology to the kinase domain of the other members of the family that have high homology. Even if this is a kinase domain, it is non-functional as it has been shown to be devoid of any significant activity. Therefore it is very unlikely for such kinase inhibitor compounds detected by this assay compounds to work through erbB-3. It is contemplated that the assay is also useful for detecting compounds that block the binding of heregulin to erbB3 or the binding of erbB3 to erbB2.

“Compound has activity through EGFR” means that proliferation is reduced as a consequence of the inhibition of an activity associated with EGFR. The invention is particularly useful for detecting compounds which are kinase inhibitors, for example ATP mimetics. It is contemplated that the assay is also useful for detecting compounds that block the binding of EGF to EGFR or binding of erbB3 to EGFR or EGFR dimerisation.

“Tested in parallel” means assayed sufficiently close in time to act as a control. For the avoidance of doubt, exactness in time is not required.

“Non-specific cell toxicity” means poisonous effects of a compound exerted through the inhibition of cellular mechanisms or processes other than those which the agent is intended to interrupt.

“erbB-2” is defined as Epidermal Growth Factor Receptor 2.

“erbB-3” is defined as Epidermal Growth Factor Receptor 3.

“EGFR” is defined as Epidermal Growth Factor Receptor 1.

“Heregulin β1” is a member of the neuregulin family of ligands that preferentially promote the formation of erbB-2/erbB-3 heterodimers.

Note that erbB family of receptor tyrosine kinases, which include EGFR, erbB-2, erbB-3 and erbB-4, are reviewed in Olayioye et at, EMBO J., 2000, 12, 3159.

“Ligand binding to erbB-3 which in turn activates erbB-2 driven cell proliferation” means, without wishing to be bound by theoretical considerations, that since no cognate ligand is known for erbB-2, this receptor can be activated indirectly by ligand binding to erbB-3 which then forms a heterodimer with erbB-2, the intracellular kinase domain thereof then being activated to start mitogenic signaling mechanisms in the cell. Compounds which bind to and inhibit the kinase domain are detected by the claimed invention.

“Immortalised normal epithelial H16N-2 cells” have been described by Band, V. and Sager, R. Tumour progression in breast cancer. In: J. S. Rhim and A. Dritschilo (eds.), Neoplastic Transformation in human Cell Culture, pp 169-178. Clifton, N.J.: Humana Press, 1991) and are obtainable from the Dana-Farber Cancer Institute, 44 Binney Street, Boston, Mass. 02115, USA. Literature references which involve use of H16N-2 cells include the following: Ram (1996) Molecular Carcinogenesis 15: 227-238; Ram (1996) Cell Growth and Differentiation 7:551-561; Ram (1995) J. Cellular Physiology 163: 589-596; Berquin (2001) 20: 4089-4028; Ram (2000) J. Cellular Physiology 183: 301-313 and Ram (2000) Cell Growth and Differentiation 11:173-183.

“Ligand for erbB-3” means a member of the neuregulin family of ligands that preferentially promotes the formation of erbB-2/erbB-3 heterodimers; heregulin β1 is preferred.

“Ligand for EGFR” means TGFα or EGF; EGF is preferred.

The invention will now be illustrated in the following non-limiting Examples in which:

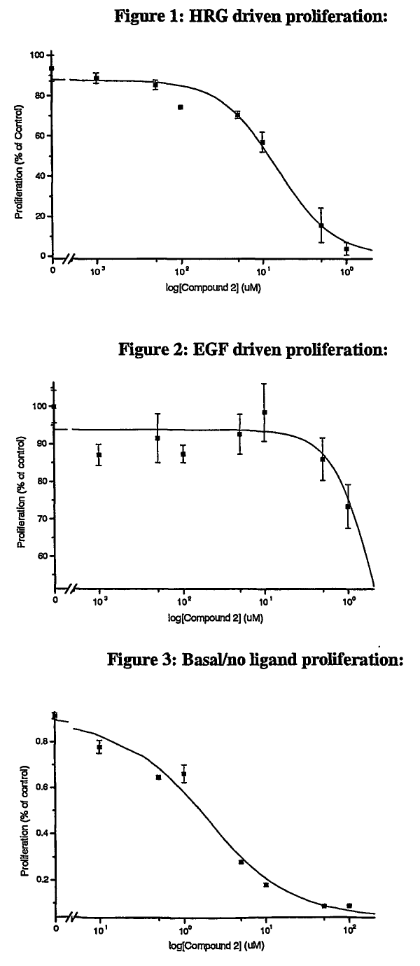

FIG. 1 shows the effect of compound 2 on heregulin driven cell proliferation

FIG. 2 shows the effect of compound 2 on EGF driven cell proliferation

FIG. 3 shows the effect of compound 2 on cells in the absence of ligand to detect non-specific toxicity.

### EXAMPLE 1

H16N-2 Cell Proliferation Assay

This assay measures the ability of a test compound to inhibit heregulin β1 or EGF driven proliferation of H16N-2 cells. These non-neoplastic eptihelial cells respond in a proliferative manner to stimulation with either EGF or heregulin β1 (Ram, G. R. and Ethier, S. P. (1996) Cell Growth and Differentiation, 7, 551-561) were isolated from human mammary tissue (Band, V. and Sager, R. Tumour progression in breast cancer. In: J. S. Rhim and A Dritschilo (eds.), Neoplastic Transformation in human Cell Culture, pp 169-178. Clifton, N.J.: Humana Press, 1991) and were obtained from the Dana-Farber Cancer Institute, 44 Binney Street, Boston, Mass. 02115.

H16N-2 cells were routinely cultured in culture medium (a 1:1 mix of Gibco F12 and Ham's αMEM media containing 1% foetal calf serum, 10 mM HEPES, 1 μg/ml Insulin, 12.5 ng/ml EGF, 2.8 μM Hydrocortisone, 2 nM Estradiol 5 μM Ascorbic Acid, 10 μg/ml Transferrin, 0.1 mM Phosphoethanolamine, 15 nM Sodium Selenite, 2 mM Glutamine, 10 nM Tri-iodo-thrynoine, 35 μg/ml Bovine pituitary Extract and 0.1 mM Ethanolamine) at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.0×10³ cells per well of a 96 well plate in the above media at 37° C. in 7.5% CO₂ and allowed to settle for 72 hours.

Following this, the cells were starved of serum for 24 hours upon the addition of starvation medium (a 1:1 mix of Gibco F12 and Ham's αMEM media containing, 10 mM HEPES, 2 nM Estradiol, 5 μM Ascorbic Acid, 10 μg/ml Transferrin, 0.1 mM Phosphoethanolamine, 15 nM Sodium Selenite, 2 mM Glutamine, and 0.1 mM Ethanolamine) and incubated at 37° C. in 7.5% CO₂. The cells were then treated with or without compound at a range of concentrations in dimethylsulfoxide (DMSO) (1% final) for two hours before the addition of exogenous ligand (at a final concentration of 100 ng/ml of heregulin β1 or 5 ng/ml of EGF) and incubation with both ligand and compound for 4 days at 37° C. in 7.5% CO₂. Following the incubation period, cell numbers were determined by addition of 50 μl of 3-(4,5-Dimethylthiazol-2-yl)-2,5-diphenyltetrazolium bromide (MTT) (stock 5 mg/ml) and incubated at 37° C. in a 7.5% CO₂ air incubator for 2 hours. MTT solution was then removed from the cells by aspiration, which were then allowed to air dry and were dissolved upon the addition of 100 μl of DMSO.

Absorbance of the solubilised cells was read at 540 nm to quantify cell biomass. Inhibition of proliferation was expressed as an IC₅₀ value. This was determined by calculation of the concentration of compound that was required to give 50% inhibition of proliferation. The range of proliferation was calculated from the positive (vehicle plus ligand) and negative (vehicle minus ligand) control values.

### EXAMPLE 2

Test Results Obtained Using the Cell Proliferation Assay of Example 1

Typical test data obtained for erbB-2 selective compounds, EGFR selective compounds and a compound with activity at both receptors is shown below along with a control (no ligand) for measurement of non-specific toxicity.

| | | Heregulin | | EGF | | Basal | |
|---|---|---|---|---|---|---|---|
| | | (Average) | | (Average) | | (Average) | |
| | Compound | IC50 | SD | IC50 | SD | IC50 | SD |
| erbB-2 | 1 | 0.054 | 0.050 | 0.945 | 0.078 | 3.954 | 3.320 |
| selectives: | 2 | 0.155 | 0.005 | >1 | 0.000 | 2.353 | 0.427 |
| | 3 | 0.432 | 0.165 | >1 | 0.000 | 4.848 | 1.746 |
| dual | 4 | 0.011 | 0.012 | 0.109 | 0.036 | 0.445 | 0.395 |
| specificity: | | | | | | | |
| EGFR | 5 | 0.009 | 0.010 | 0.047 | 0.067 | 0.371 | 0.195 |
| selectives: | | | | | | | |

“SD” means standard deviation.

The plots, which were used to generate the summary data above, are shown in FIGS. 1-3.

1. A cellular proliferation assay for a compound acting through erbB-2 which comprises:
i) providing a cell comprising erbB-2 and erbB-3 and which is responsive to ligand stimulated cell proliferation under conditions suitable for cell proliferation;
ii) providing a first ligand which is a ligand for erbB-3 capable of inducing proliferation by the cell;
iii) contacting i) and ii) in the presence and absence of a compound; and
iv) determining whether the compound has any effect on reducing cell proliferation.

2. A cellular proliferation assay according to claim 1 comprising the addition of a control wherein the compound is tested in parallel in the absence of ligand to detect non-specific cell toxicity.

3. A cellular proliferation assay according to claim 1 or 2 further comprising:
i) providing a second cell comprising erbB-2, erbB-3 and EGFR under conditions suitable for cell proliferation;
ii) a second ligand which is a ligand for EGFR capable of inducing proliferation by the second cell;
iii) contacting i) and ii) in the presence and absence of a compound; and
iv) determining whether the compound has any effect on reducing cell proliferation.

4. A cellular proliferation assay according to claims 1 or 2 wherein the cell endogenously expresses EGFR and/or erbB-2 and/or erbB-3.

5. A cellular proliferation assay according to claim 4 wherein the cell endogenously expresses EGFR, erbB-2 and erbB-3.

6. A cellular proliferation assay according to claim 1 wherein the first ligand is a heregulin.

7. A cellular proliferation assay according to claim 6 wherein the first ligand is heregulin β1.

8. A cellular proliferation assay according to claim 3 wherein the second ligand is EGF.

9. A cellular proliferation assay according to claims 1 or 3 wherein the cell is an immortalised normal epithelial H16N-2 cell.

10. A cellular proliferation assay according to claims 1 or 3 wherein the cell is a MCF-7 cell.
